(19) United States
(12) Patent Application Publication
(10) Pub. No.: US 2006/0270949 A1
(43) Pub. Date: Nov. 30, 2006

(54) Monitoring apparatus for ambulatory subject and a method for monitoring the same

(76) Inventors: Merryn Joy Mathie, Syndey (AU); Branko G. Celler, Sydney (AU); Nigel H. Lovell, Sydney (AU)

Correspondence Address: PARK LAW FIRM, 3255 WILSHIRE BLVD, SUITE 1110, LOS ANGELES, CA, 90010, US

(21) Appl. No.: 10/568,511

(22) PCT Filed: Aug. 13, 2004
(86) PCT No.: PCT/AU04/01081

§ 371 Date: Feb. 15, 2006

Related U.S. Application Data
(60) Provisional application No. 60/514,969, filed on Oct. 27, 2003.

(30) Foreign Application Priority Data
Aug. 15, 2003 (AU) 2003904336

(51) Int. Cl. A61B 5/103; G08B 23/00

(52) U.S. Cl. 600/595; 340/573.7

(57) ABSTRACT

The invention provides a monitoring apparatus (1) for an ambulatory subject (2) that includes a portable monitor (3) mountable on subject (3) that includes an accelerometer that determines the instant acceleration of subject (2) in one or more determined directions. It further includes a processing unit (7) that determines at least one instant ambulatory performance indicia of subject (2) from the determined instant acceleration, determines at least one designated performance threshold from at least one previously determined instant ambulatory performance indicia and initiates an event under predetermined conditions that is communicated to remote receiver (5) for the attention of carer (11), providing remote monitoring and supervision of subject (2) based on ambulatory performance of subject (2).

### FIELD OF THE INVENTION

The invention is in the field of monitoring methods and apparatus for ambulatory subjects.

### PRIOR ART

In accordance with the statistics in some countries the population is ageing and it is projected that by the year 2051 those aged 65 years and over will constitute approximately one quarter of the total population.

Falls are one of the greatest risks facing this group and in the over 65 age group, accidents are the fifth highest cause of death, and approximately two thirds of accidents are falls. Falls also account for more than half of all injury-related hospital admissions in this group.

Falls and collapse are associated with functional decline, leading to disability, dependence and nursing home placement, even in cases where the fall did not cause injury. Up to half of all older people who fall or collapse without suffering injuries are unable to get up without assistance.

In the case of the elderly or infirm persons living alone, an inability to rise can lead to serious consequences of extreme distress, muscle damage, pneumonia, pressure sores, dehydration, hypothermia and mortality. Many such people become afraid and so restrict their daily activities and exercise, which in turn leads to a further reduction in health and wellbeing.

Some personal alarm systems provide such venerable people with an emergency button however this technology is rendered ineffective if the person is unable to press the button due to unconsciousness, injury or immobility.

Furthermore the ageing population and the related increasing prevalence of chronic disease are placing a large burden on the hospital system. There is a need to provide alternatives to hospital care for these patients.

One of the most important considerations in independent living is functional status; that is, the ability of a person to carry out routine daily tasks in his or her normal (home) environment. There are many different measurements that provide indication of functional status. These include, but are not limited to, the time taken to rise from sifting, postural sway when standing, walking speed, and step rate variability. Traditionally, these parameters have been measured in a dedicated laboratory in an expensive, time-consuming procedure, or they have been measured subjectively in the clinic or home using clinician observation or patient recall.

It is therefore an object of the invention to overcome some of the problems of the prior art or at least to provide a useful alternative.

### SUMMARY OF THE INVENTION

One aspect of a preferred embodiment of the invention provides a monitoring apparatus for an ambulatory subject including:
a portable monitor mountable on the subject that includes an accelerometer that determines the instant acceleration of the subject in one or more determined directions;
a processing unit that:
   a) determines at least one instant ambulatory performance indicia of the subject from at least one determined instant acceleration of the subject in one or more instants of time;
   b) determines at least one designated performance threshold from at least one previously determined instant ambulatory performance indicia;
   c) determines if the subject's instant ambulatory performance indicia is below or above the at least one designated performance threshold;
   d) initiates at least one event if the determined instant ambulatory performance indicia is above or below the determined at least one designated performance threshold; and
a communications unit that communicates an initiated event to a remote receiver.

Preferably the at least one designated performance threshold is determined by the processing unit from a plurality of previously determined instant ambulatory performance indicia.

Preferably the at least one event is initiated only if the determined instant ambulatory performance indicia is below or above the determined at least one designated performance threshold for a designated period of time.

Preferably the designated first period of time is determined from a plurality of previously determined instant ambulatory performance indicia.

Preferably the accelerometer simultaneously determines the acceleration of the subject in three orthogonal directions.

Preferably the portable monitor is configured to be mounted on an upright ambulatory such that one of the three orthogonal directions is in a vertical direction or within a designated angle of the vertical direction.

Preferably an initiated event is communicated by the apparatus to the remote receiver by wireless communication.

In another embodiment it is also preferred:
a first instant ambulatory performance indicia representative of movement activity in the subject is determined from the instant magnitude of the sum of the instant acceleration of the subject in one or more determined directions;
a first acceleration threshold magnitude that is representative of a lack of normal expected subject movement is designated as a first designated performance threshold;
a first event representative of ‘an absence of a normal amount of movement in the subject indicative of a possible inability to rise due to a collapse or other adverse event’ is initiated if the determined first instant ambulatory performance indicia is below the first designated acceleration threshold magnitude for a first designated period of time.

It is also further preferred:
a second instant ambulatory performance indicia representative of the instant cranio-caudal angle of the subject relative to an upright disposition of the subject, is determined from at least one of the determined instant acceleration of the subject in one or more determined directions;
an angle magnitude that is representative of a cranio-caudal angle of the subject relative to an upright subject where the disposition of the subject is deemed to be no longer upright is designated as a second designated performance threshold;
a second acceleration threshold magnitude representative of an abnormally high subject movement is designated as a third designated performance threshold; and
a second event representative of an abnormal acceleration of the subject followed by a laying down subject disposition indicative of a possible fall coupled with a subsequent absence of getting up from the laying down disposition indicative of a possible debilitating fall is initiated if:
   a) the determined first instant ambulatory performance indicia is above the second designated acceleration threshold for a second designated period of time; and
   b) within a third designated period of time of the end of the second designated period of time the determined second instant ambulatory performance indicia is greater than the designated angle threshold; and then
   c) the determined first instant ambulatory performance indicia is below the first designated acceleration threshold magnitude for a first designated period of time.

Another aspect of the invention provides a method of monitoring an ambulatory subject including:
mounting a portable monitor on the subject that is in wireless communication with a remote receiver, wherein the monitor includes an accelerometer to determine simultaneously the instant acceleration of the subject in at least three different directions at different instants in time;
determining a plurality of instant ambulatory performance indicia based on the determined instant acceleration of the subject;
determining at least one designated performance threshold corresponding to the each ambulatory performance indicia from at least one previously determined corresponding instant ambulatory performance indicia;
determining if the subject's instant ambulatory performance indicia is below or above the corresponding at least one designated performance threshold and initiating a corresponding event;
communicating a designated initiated event to the remote receiver.

Preferably the at least one designated performance threshold is determined from a plurality of previously determined instant performance indicia, and the designated performance threshold can responsively and cooperatively adapt to statistical changes in previously determined instant performance indicia over time.

Preferably the event is initiated only if the determined instant ambulatory performance indicia is below or above the determined at least one designated performance threshold for a designated period of time.

Another aspect of the invention provides a monitoring apparatus for an ambulatory subject including:
a portable monitor mountable on the subject that includes an accelerometer that simultaneously determines the instant acceleration of the subject in at least three different directions;
a processing unit that:
   a) determines the instant magnitude of the sum of the instant acceleration of the subject in the at least three different directions;
   b) determines if the determined instant magnitude does not exceed a first designated acceleration threshold magnitude for a first designated period of time, where the first designated acceleration threshold magnitude is representative of a lack of normal expected subject movement;
   c) initiates an event representative of an absence of a normal amount of movement in the subject indicative of a possible inability to rise due to a collapse or other adverse event if the determined instant magnitude does not exceed the first designated acceleration threshold magnitude for at least the first designated period of time; and
a communications unit that communicates an initiated event to a remote receiver.

In this aspect of the invention it is preferred:
the processing unit:
   a) determines if the determined instant magnitude of the sum of the instant acceleration of the subject in the at least three different directions exceeds a second designated acceleration threshold magnitude for at least a second designated period of time;
   b) determines the magnitude of the instant angle of the subject being the magnitude of the angle between the cranio-caudal axis of the subject and the cranio-caudal axis of the subject when in an upright disposition from at least one of the determined instant acceleration of the subject in one or more determined directions;
   c) determines if the determined instant angle of the monitor is greater or less than a designated angle magnitude threshold; and
   d) initiates an event representative of an abnormally high acceleration of the subject followed by a laying down subject disposition indicative of a possible fall coupled with a subsequent absence of getting up from the laying down disposition indicative of a possible debilitating fall if:
      I. the determined instant magnitude of the sum of the instant acceleration of the subject in the at least three different directions exceeds the second designated acceleration threshold magnitude for at least a second designated period of time; and
      II. within a third designated period of time after the end of the second designated period of time the determined instant angle of the subject is greater than the designated angle magnitude threshold; and
      III. within the third designated period of time after the end second designated period of time the instant magnitude of the sum of the instant acceleration of the subject in the at least three different directions does not exceed a third designated acceleration magnitude for at least a fourth designated period of time.

Preferably the accelerometer simultaneously determines the acceleration of the subject in three orthogonal directions.

Preferably the portable monitor is mounted on an upright subject in an orientation so that one of the three orthogonal directions is in a vertical direction or within a designated angle of the vertical direction.

In another aspect the invention provides a method for detecting a person's inability to rise after a fall, collapse or other adverse event using a triaxial accelerometer included in a personal wearable ambulatory monitoring device. The first part of the procedure involves the detection of an inability to rise caused by a fall event. The first step in the method is sampling an output from the triaxial accelerometer that is indicative of body acceleration and body angle. The next step is to determine whether a fall has taken place by comparing the magnitude of the acceleration vector to an acceleration magnitude threshold for a period equal to a time duration threshold to determine the presence of an abnormal acceleration. If an abnormal acceleration is detected then the body angle is compared to a threshold value to identify a body state indicative of lying. A subsequent absence of movement is detected by comparing the magnitude of the acceleration vector to a second acceleration magnitude threshold. The second part of the procedure involves the detection of an inability to rise due to collapse or other adverse event. The first step in the method is sampling an output from the triaxial accelerometer that is indicative of body acceleration and body angle. The next step is to identify an inability to rise by comparing the magnitude of the acceleration vector to an acceleration magnitude threshold for a period equal to a time duration threshold to determine the absence of a normal amount of movement.

In another aspect of the invention it provides a method for monitoring a person's movement to detect an inability to rise due to a fall through using a triaxial accelerometer included in a personal monitoring system that consists of a receiver unit and a personal monitoring device, which communicates with the receiver units by means of wireless communication, and which is configured to be carried on the person and containing the triaxial accelerometer, including the steps of:
(a) sampling an output from the triaxial accelerometer indicative of body acceleration and body angle;
(b) comparing the magnitude of the acceleration vector to an acceleration magnitude threshold for a period equal to a time duration threshold to determine the presence of an abnormal acceleration indicative of a fall;
(c) identifying a body state indicative of lying by comparing the body angle to a threshold value;
(d) determining the subsequent absence of movement by comparing the magnitude of the acceleration vector to a second acceleration magnitude threshold.

Preferably a severe fall is signalled via a communications network when a severe fall has taken place.

In another aspect the invention provides a method for monitoring a person's movement to detect an inability to rise due to a collapse or other adverse event using a triaxial accelerometer included in a personal monitoring system that consists of a receiver unit and a personal monitoring device, which communicates with the receiver units by means of wireless communication, and which is configured to be carried on the person and containing the triaxial accelerometer, including the steps of:
(a) sampling an output from the triaxial accelerometer indicative of body acceleration and body angle;
(b) comparing the magnitude of the acceleration vector to an acceleration magnitude threshold for a period equal to a time duration threshold to determine the absence of a normal amount of movement.

Preferably the method further includes the step of signalling the extended absence of movement via a communications network when no movement has occurred for the specified period.

In another aspect the invention provides longitudinal tracking of clinically significant parameters to detect early changes in functional status including but not limited to the onset of near falls and stumbles, clinically significant parameters are extracted from the data obtained from the triaxial accelerometer and stored in a database, deviations from the statistical normal values for each parameter are flagged, an alert is generated each time that a deviation is flagged, long term trends in the parameters are also flagged, and when a trend is detected in a parameter that exceeds a change threshold an alert is generated.

### DRAWINGS

The invention will now be described by way of example only with reference to the following drawings in which:

FIG. 1: illustrates a schematic view of a preferred embodiment of a monitoring apparatus for an ambulatory subject in accordance with the invention;

FIG. 2: illustrates a schematic view of a preferred embodiment of the portable monitor in FIG. 1 with one of three sensitive axis aligned in a vertical direction;

FIG. 3: illustrates an example of a net measured acceleration vector using the portable monitor in FIG. 2 resolved into an acceleration vector component along one of three sensitive axis of the portable monitor.

FIG. 4: illustrates an example of a net measured acceleration vector using the portable monitor in FIG. 2 resolved in to a gravity acceleration vector component, and a subject movement vector component which is representative of the subject's horizontal and vertical movements contribution to the net measured acceleration vector.

FIG. 5: illustrates a schematic view of the subject in FIG. 1 in a partly non upright disposition;

FIG. 6: illustrates a schematic view of the portable monitor in FIG. 1 in the same disposition as the portable monitor and subject in FIG. 5;

FIG. 7: illustrates a schematic view of a preferred embodiment of a model for classifying a range of ambulatory movements of a monitored subject in the form of a decision making tree, suitable for use with a preferred embodiment of the invention;

FIG. 8: illustrates an illustrative example of longitudinal monitoring of a parameter representative of ambulatory movement of a subject in accordance with a preferred embodiment of the invention;

FIG. 9: illustrates a schematic view of a preferred embodiment of a processing model for monitoring a subject to detect whether the subject failed to get up from a non upright disposition either for a designated period of time, or pursuant to a fall event in accordance with a preferred embodiment of the invention.

### DETAILED DESCRIPTION OF EMBODIMENTS OF THE INVENTION

It is to be noted where possible features common to the various embodiments illustrated in the figures are referred to in each drawing by a respective common feature number.

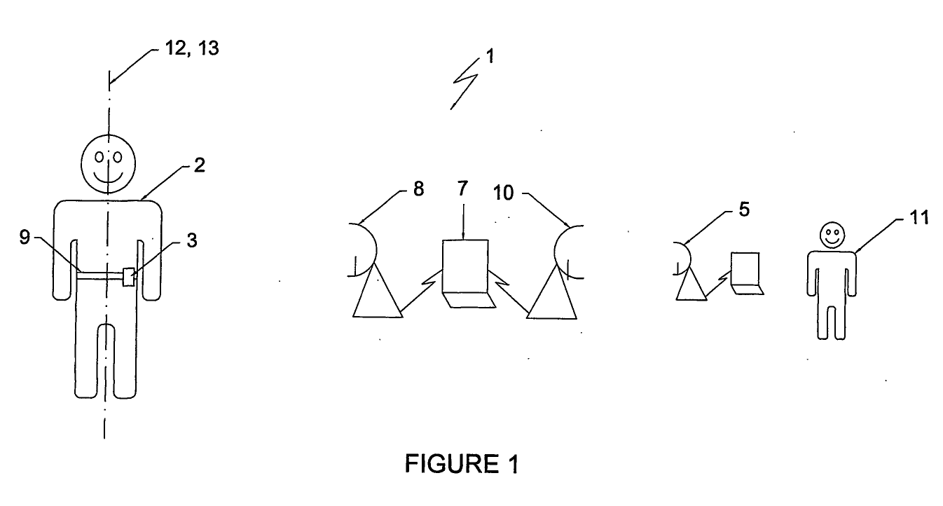

A preferred embodiment of a monitoring apparatus 1 for an ambulatory subject 2 in accordance with the invention will be described with general reference to FIGS. 1 to 6, and for description purposes the preferred embodiment will be described in the context of subject 2 being monitored by carer 11 being remote from subject 2, but in cooperative operable communication with apparatus 1 via receiver 5.

Apparatus 1 includes portable monitor 3 mountable on subject 2, mounted for example on belt 9 worn by subject 2 by a belt clip attached (not shown) to monitor 3.

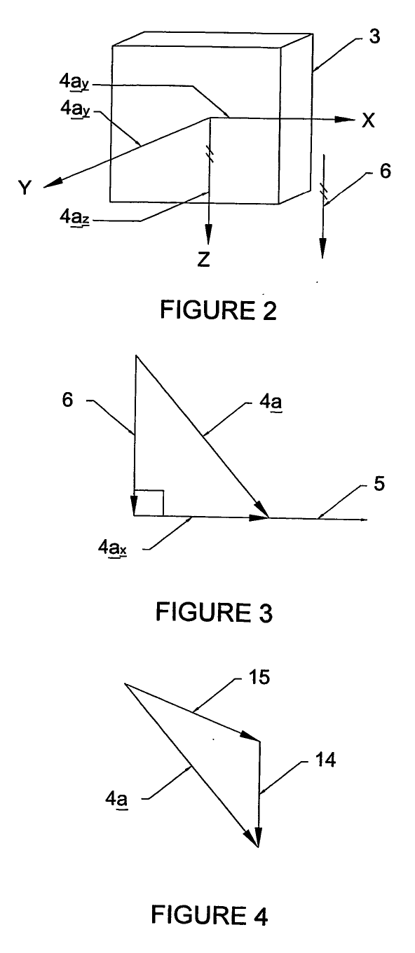

Monitor 3 includes a three dimensional accelerometer to measure simultaneously instant acceleration vectors 4aₓ, 4a_{y}, 4a_{z} of subject 2 in three different orthogonal directions X, Y, Z as shown in FIG. 2.

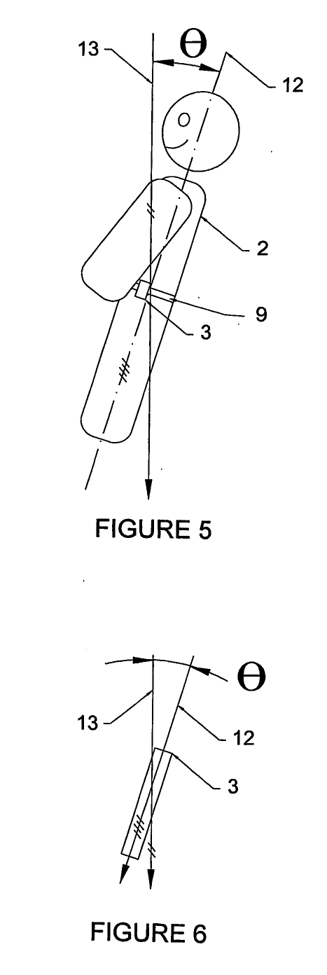

Monitor 3 is arranged in relation to its mounting belt clip (not shown) so that its sensitive measurement axis in direction Z is orientated to be in a vertical direction 6 as shown in FIG. 2 or near vertical (not shown) when monitor 3 is mounted on upright subject 2 via its belt clip. In this orientation acceleration vector 4a_{z} provides a reference for calculating the instant body angle Θ of subject 2 shown in FIG. 5 as the angle between the cranio-caudal axis 12 of subject 2 relative to an upright disposition of subject 2 shown as vertical axis 13.

Apparatus 1 further includes data processing and storage unit 7 for processing the measured simultaneous instant acceleration vectors 4aₓ 4a_{y} 4a_{z} at different instants in time, initiating events for the attention of the carer according to the determinations of the data processing. Data processing and storage unit 7 also logs and stores the processed data for use and or reference by apparatus 1 and or carer 11 as required.

Events that are initiated by data processing and storage unit 7 are communicated to carer 11 for attention by data processing and storage unit 7 via the operationally cooperative pair of transmitter 10 and receiver 5. In this way remote supervisory monitoring of the ambulatory status and performance of subject 2 can be achieved by carer 11.

Data processing and storage unit 7 in FIG. 1 is shown to be remote from portable monitor 3 but in wireless communication with data processing and storage unit 7 via local receiver/transmitter 8. However data processing and storage unit 7 may in the alternative be in part or in whole integrated (not shown) with portable monitor 3.

While the communication between data processing and storage unit 7 and remote receiver 5 are shown in FIG. 1 to be in wireless communication, the communication may be by any suitable communication mode and in the case of the embodiment of data processing and storage unit 7 shown in FIG. 1 a suitable alternative communication mode between data processing and storage unit 7 and remote receiver 5 for example may be a wired telephone network (not shown) or a combination of wired and wireless telephone network, radio communication or any other wireless communication technology.

In another embodiment, monitor 3 identifies the methods of transmission that are available from its current location to data processing and storage unit 7. These may include, but are not limited to wireless network protocols, and mobile telephony protocols. The monitor 3 then selects the most appropriate method of transmission and transmits the data to the data processing and storage unit 7 using this method. If no method of transmission is available then the data are retained in monitor 3 unit until a method of transmission becomes available.

In another embodiment monitor 3 may also include a geographical positioning system (GPS) and in the case of an event being initiated the GPS coordinates of monitor 3 can be communicated to receiver 5 with the event which provides the location of subject 2.

Data processing and storage unit 7 includes a suite of data processing models (generally not shown) each processing model representative of a different ambulatory performance indicia of interest to carer 11 for the proper remote supervisory ambulatory monitoring of subject 2.

The data input in to each of the processing models is from the instant acceleration vectors 4aₓ, 4a_{y}, 4a_{z} representative of the movement dynamics of subject 2 measured by portable monitor 3. The processing model inputs may be one vector 4aₓ, 4a_{y}, 4a_{z} component or a combination of vector 4aₓ, 4a_{y}, 4a_{z} components.

Other inputs to the processing models include inputs calculated by the processing models themselves from one or more vector 4aₓ, 4a_{y}, 4i a_{z} component inputs. Such calculated processing model inputs include calculated performance threshold magnitudes for the ambulatory performance indicia, calculated instant body angle Θ.

An output of each processing model is one instant ambulatory performance indicia of subject 2 determined from the instant acceleration vectors 4aₓ, 4a_{y}, 4a_{z} and the requirements of the processing model.

The calculated instant ambulatory performance indicia is logged and stored in data processing and storage unit 7 for further use and or reference by apparatus 1 and or carer 11 as required.

Another output of each processing model is calculated performance threshold magnitudes for the corresponding ambulatory performance indicia.

Such performance threshold magnitudes are calculated from the ambulatory performance indicia logged and stored during the absence of adverse events and the ambulatory performance indicia logged and stored provide a convenient and in-situ reference to calculate suitable performance threshold magnitudes for the ambulatory performance indicia. Such performance threshold magnitude calculations maybe based on for example, statistical techniques like data averaging by calculating root means square values of sampled historical data. The performance threshold magnitude calculations for a specific processing model is done by the processing model itself based on ambulatory performance indicia determined by the model itself.

The performance threshold magnitudes for the ambulatory performance indicia once calculated are then used as calculated inputs into the respective processing model to provide a reference to the processing model to initiate an event which is another calculated type output of the processing model.

An event is initiated when the instant ambulatory performance indicia is above or below the respective threshold for a designated period of time, which may be one instant or longer, as required by the processing model. The initiated event if required by the processing model is then communicated by apparatus 1 to carer 11 for attention.

Furthermore the processing model is adapted so that the performance threshold magnitudes inputted to the processing model are updated by the processing model itself as the calculated thresholds magnitudes vary with time. In this way the threshold magnitudes used by the processing model adapt to the longer term ambulatory performance changes in subject 2 which are reflected in the changes in the calculated threshold magnitudes over time.

Of course the apparatus 1 provides for performance threshold magnitudes to be inputted by human intervention into the processing model as required.

A calculated output of the processing model is the calculated instant body angle Θ of subject 2 shown in FIG. 5. The instant body angle Θ is the inverse cosine of the magnitude of the acceleration vector 4a_{z} when the monitor 3 is mounted on upright subject 2 so that so that the direction of acceleration vector 4a_{z} is orientated to be in a vertical direction 6 as shown in FIG. 2 or near vertical.

The calculated instant body angle Θ of subject 2 is useful parameter and is used as a calculated input in the processing model to help discriminate between different ambulatory activities, subject dispositions, and transitions between different dispositions.

The instant body angle Θ of subject 2 as shown in FIG. 5 can be calculated from the instant acceleration vectors 4aₓ, 4a_{y}, 4a_{z}, for example by sampling the magnitude of the acceleration vectors 4aₓ, 4a_{y}, 4a_{z}, at 40 Hz and then filtering the measured instant acceleration vectors 4aₓ, 4a_{y}, 4a_{z} samples with a low pass filter with a 3 dB cut-off between 0.1 and 0.5 Hz. The resultant magnitude of this filtered sample is representative of the magnitude of the acceleration measurement due to gravity (with the contributions due to movement in subject 2 having been filtered out by the low pass filter process) but resolved in the direction of the instant cranio-caudal axis 12 as disposed in FIGS. 5 and 6. The inverse cosine of the resultant magnitude of this filtered sample is a measure of body angle Θ.

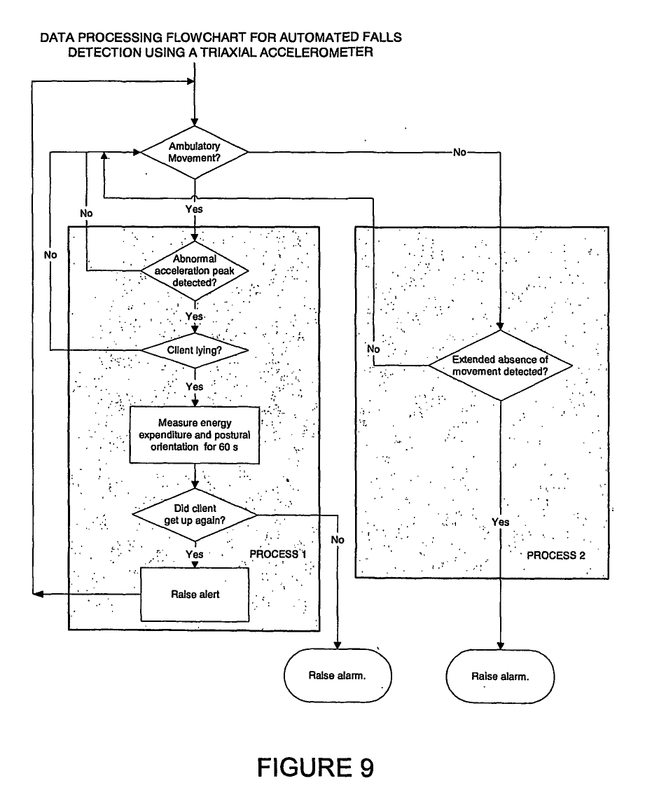

By way of example, one useful ambulatory performance indicia of subject 2 of interest to carer 11 would be whether subject 2 indicates an absence of normal movement for a prolonged period of time which can be indicative of an inability of subject 2 to rise due to some adverse event. A schematic model of this is illustrated in a general way in FIG. 9 and identified as process 2 in that schematic model.

The ambulatory performance indicia may be characterised by designating an acceleration threshold magnitude which is representative of a minimum expected level of movement in subject 2 under non adverse or normal conditions, for example during sleep.

In this example the instant magnitude of the sum of the instant acceleration vectors 4aₓ, 4a_{y}, 4a_{z}, is measured my monitor 3 and the root mean square value of the instant magnitude is calculated every minute and compared to the designated acceleration threshold magnitude. If the root means square value of the instant magnitude remains below the designated acceleration threshold magnitude for a designated period of time, for example 6 hours then this state of affairs would be of concern to carer 11 and consequently an event is initiated by the apparatus 1 representative of this state of affairs and communicated to carer 11 for their attention and or action as required.

The designated acceleration threshold magnitude may be set by a reference to previous instant acceleration vector 4aₓ, 4a_{y}, 4a_{z} data that has been logged and stored for subject 2 gathered during specific sleep tasks given to subject 2 under non adverse conditions, or gathered from subject 2 normal sleeping routine.

That is for example the root mean square of the instant magnitude of the sum of the instant acceleration vectors 4aₓ, 4a_{y}, 4a_{z} is calculated over a period during which subject 2 is known to be in normal sleep, and that root mean square value is used as the designated acceleration threshold magnitude in the processing model for the ambulatory performance indicia of interest.

Preferably the designated acceleration threshold magnitude in the processing model is adapted to contingently vary over time as the calculated root mean square of the instant magnitude of the sum of the instant acceleration vectors 4aₓ, 4a_{y}, 4a_{z} value during normal sleep changes over time as the normal sleeping patterns of movements of subject 2 changes.

In this way the processing model is adaptive to the changing sleeping conditions of subject 2 but can discriminate relatively short term variances which may indicate an adverse event such as an absence of normal movement for a prolonged period of time which can be indicative of subject 2 inability to rise due to the adverse event.

By way of another example, another useful ambulatory performance indicia of subject 2 of interest to carer 11 would be whether subject 2 fails to get up after an abnormally high movement which can be indicative of subject 2 inability to rise after a severe fall or collapse. A schematic model of this is illustrated in a general way in FIG. 9 and identified as process 1 in that schematic model.

The ambulatory performance indicia may be characterised by designating several threshold magnitudes.

A first acceleration threshold magnitude is designated which is representative of a minimum expected level of movement in subject 2 under non adverse conditions for when subject 2 is going from a disposition of lying down to a disposition of getting up.

A second acceleration threshold magnitude is designated which is representative of a maximum expected level of movement in subject 2 under non adverse conditions for when subject 2 is going from a disposition of being upright to a disposition of lying down. For example a nominal value maybe 1.8 g where g is the acceleration due to gravity, approx. 9.81 m.s⁻².

Both these thresholds may be set by a reference to previous instant acceleration vector 4aₓ, 4a_{y}, 4a_{z} data that has been logged and stored for subject 2 gathered during specific getting up lying, down tasks given to subject 2 under non adverse conditions, or gathered from subject 2 normal daily routine.

A body angle threshold is designated where for convenient reference the body angle Θ as shown in FIG. 5 is a measure of the angle between a cranio-caudal axis 12 of subject 2 and vertical axis 13.

The body angle threshold being representative of an angle when subject 2 is considered to no longer be upright, for example a body angle in excess of 60 degrees (form the vertical) may be deemed to be no longer upright.

In this example the sum of the simultaneously determined instant acceleration vectors 4aₓ, 4a_{y}, 4a_{z} are processed into an instantaneous polar acceleration vector with polar coordinates and the magnitude of this polar acceleration vector is compared to the second acceleration threshold magnitude.

If the magnitude of this instant polar acceleration vector is greater than the second acceleration threshold magnitude for a minimum designated period of time then an abnormally large acceleration has been detected. In that case calculate the instant body angle Θ of subject 2 from the determined instant acceleration vectors 4aₓ, 4a_{y}, 4a_{z}, as described previously herein.

If the body angle body angle Θ of subject 2 is less than the designated body angle threshold then the previously detected abnormally large acceleration is deemed to have been attributable to a stumble or knock rather than a potential fall.

However if the instant body angle body angle Θ of subject 2 is more than the designated body angle threshold then the previously detected abnormally large acceleration is deemed to have been attributable to a fall and if the instant body angle body angle Θ of subject 2 remains more than the designated body angle threshold for more than a minimum designated period of time, then it is deemed that subject 2 is unable to get up unaided from the laying disposition perhaps a result of a debilitating fall, and a corresponding event is communicated to the carer 11 for attention.

However if the if the instant body angle body angle Θ of subject 2 does not remain more than the designated body angle threshold for more than a minimum designated period of time, then it is deemed that subject 2 was able to get up from the laying disposition and immediate assistance may not be required.

In a preferred alternative after the fall has been determined if the magnitude of the instant polar acceleration vector remains below the designated first acceleration threshold magnitude for a minimum designated period of time, then it is deemed that subject 2 is unable to get up unaided from the laying disposition perhaps a result of a debilitating fall, and a corresponding event is communicated to the carer 11 for attention.

By way of another example, other useful ambulatory performance indicia of subject 2 of interest to carer 11 would be indicia which are representative of the functional status of subject 2. Advantageously monitoring apparatus 1 provides for the longitudinally monitoring of the functional status of subject 2 with automated alarm generation when deterioration in functional status of subject 1 is detected.

For example movements such as sitting down into a chair, rising from a chair, lying down, standing up from lying and walking are all important for basic assessment of functional status. Additional or different movements may be monitored if they provide useful information relevant to a particular subject. As an example, monitoring tremor may be useful if subject 2 suffers from Parkinson's disease.

Relevant ambulatory performance indicia to these activities can be determined from the data provided by monitor 3 using modelling techniques. However before these performance indicia can be determined from the determined instant acceleration vectors 4aₓ, 4a_{y}, 4z, the subject's movement must be identified and classified to determine which model or algorithm can be applied to determine the ambulatory performance indicia.

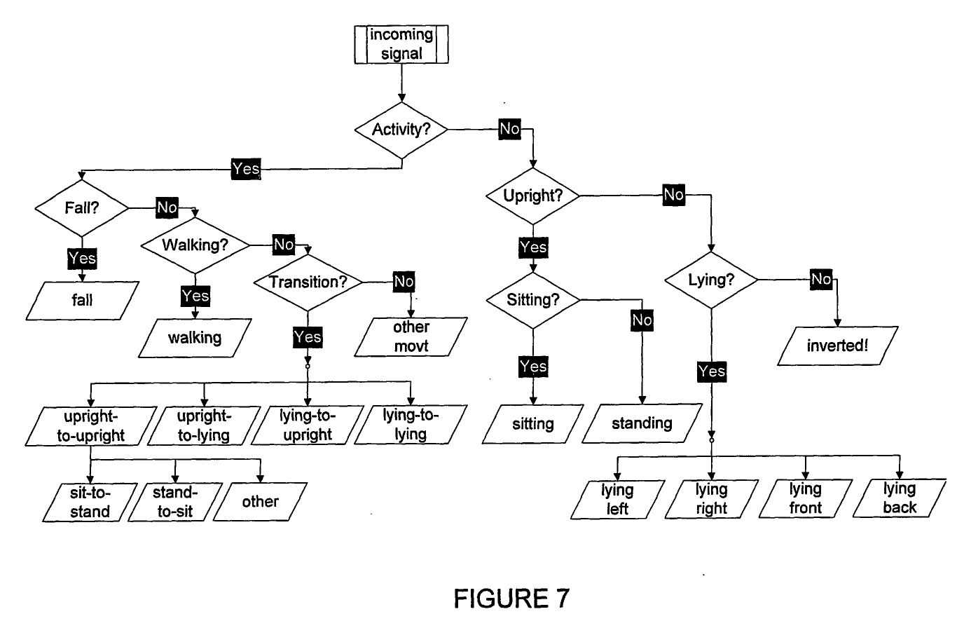

FIG. 7 illustrates a schematic view of a preferred embodiment of a model for classifying a range of ambulatory movements of a monitored subject in the form of a decision making tree, suitable for use with apparatus 1. This classification may alternatively be achieved using a range of other methods, including but not limited to, pattern recognition, neural networks, expert systems, fuzzy logic systems, by a combination of methods, or by asking the subject to carry out a particular movement so that the activity is known as a priori.

Whether the subject 2 is engaged in activity or rest ca be determined from the instant acceleration vectors 4aₓ, 4a_{y}, 4a_{z}, for example by sampling the magnitude of the acceleration vectors 4aₓ, 4a_{y}, 4a_{z}, at 40 Hz and then filtering the measured instant acceleration vectors 4aₓ, 4a_{y}, 4a_{z} samples with a high pass filter with a 3 dB cut-off between 0.1 and 0.5 Hz. The resultant magnitude of this filtered sample is representative of whether subject 2 is engaged in activity or is at rest. The algorithm disclosed by Mathie et al. which is incorporated herein by reference [Mathie, M. J., Coster, A. C. F., Lovell, N. H. and Celler, B. G. (2003). Detection of daily physical activities using a triaxial accelerometer, Medical and Biological Engineering and Computing] may be used for this purpose.

Once movements are identified, movement relevant performance indicia sensitive to functional status of subject 2 can be determined from the determined instant acceleration vectors 4aₓ, 4a_{y}, 4a_{z} by the appropriate corresponding algorithms and models.

For example:
the duration of an activity can be determined by counting the number of samples that were measured during the period of activity and dividing by the sampling rate;
the walking step rate can be determined by identifying periodic peaks in the walking signal and the walking step rate variability can be computed by comparing the timing between consecutive steps;
the postural sway amplitude can be obtained by measuring amplitude acceleration during the period of standing and frequency can be obtained by identifying a frequency spike in the Fourier transform of the signal.

In addition, general measures of movement, such as metabolic energy expenditure, can also be computed from the signals obtained from the triaxial accelerometer as metabolic energy expenditure is linearly related to the sum of the integrals of the magnitudes of the three orthogonal acceleration signals.

The data from the monitor 1 is transmitted to data processing and storage unit 7 and the sensitive relevant movement performance indicia are stored. These indicia are tracked longitudinally. Each time new data are received the data processing and storage unit 7 compares the new data to the existing data.

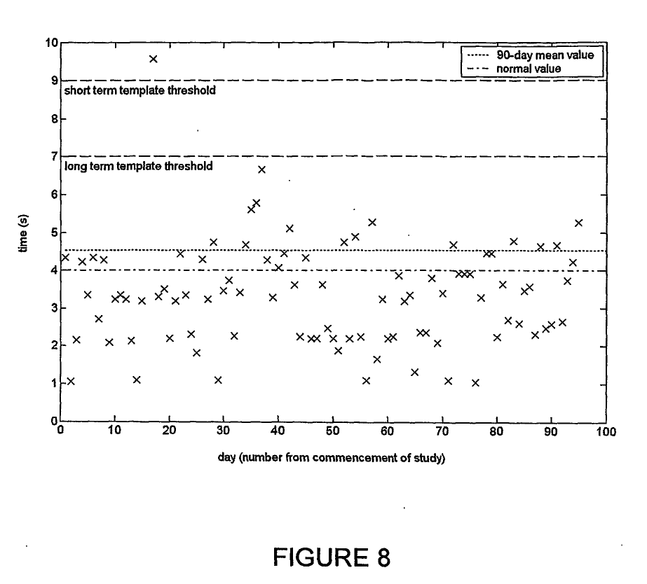

The data processing and storage unit 7 also examines the time-trend produced by the pre-existing data and the new data for example as illustrated in FIG. 8. If the value of the new data exceeds a preset threshold value, or the longitudinal trend exceeds a preset threshold (possibly either with respect to the slope of the trend, or the number of samples exceeding a preset threshold) then an alarm is raised by the data processing and storage unit 7.

The generated alarm is communicated to a carer 11, clinician, emergency call centre, and/or to the subject 2 using an appropriate telecommunications technology, including, but not limited to, telephone, facsimile, email, text message system on mobile telephone network, or an Internet alert message.

The threshold and alarm values are set so that genuine deterioration in functional ability is detected. The intent is to detect deterioration early in the process so that preventative interventions can occur to prevent morbidity. The threshold values are subject 2 specific.

During the first few weeks of use of the apparatus 1 by subject 2 the alarms are de-activated and the thresholds automatically adapt, based on the data obtained from subject 2. Following this, the thresholds are fixed by the apparatus 1, although they can still be changed manually and the alarms are enabled.

In another preferred embodiment of the invention the subect wears a triaxial accelerometer on the waist and data are sampled from the triaxial accelerometer by a microprocessor in the monitoring device and stored in a buffer in the monitoring device and the data are processed according to process 1 and process 2 following and illustrated in (FIG. 9), where process 1 relates to the detection of an inability to rise precipitated by a fall event, and process 2 relates to the detection of an inability to rise other than due to a fall event.

Process 1

1. Data are converted from Cartesian to polar coordinates;
2. The value of the polar magnitude vector is compared to a magnitude threshold (for example 1.8 g);
3. If the value of the polar magnitude is less than the value of the magnitude threshold, then continue monitoring from the start of the process 1;
4. If the value of the polar magnitude exceeds the value of the magnitude threshold, then continue monitoring the next samples;
5. If the value of the polar magnitude does not continuously exceed the value of the magnitude threshold for a period of time that exceeds a time threshold then continue monitoring from the start of process 1;
6. If the value of the polar magnitude continuously exceeds the value of the magnitude threshold for a period of time that exceeds a time threshold then an abnormally large acceleration has been identified;
7. Measure the body angle immediately following the abnormally large acceleration event;
8. Compare the body angle to a threshold angle that is indicative of a lying state;
9. If the body angle does not exceed the lying state threshold then the abnormally large acceleration event was deemed to have been a stumble or knock, but not a fall then log the event and continue monitoring the next sample from the start of process 1; otherwise
10. If the body angle does exceed the lying state threshold then the abnormally large acceleration event was deemed to have been a fall, then wait 60 s;
11. Measure the body angle again;
12. Compare the new body angle to the lying state threshold;
13. If the body angle does not exceed the lying state threshold then the person was able to rise unaided and raise a corresponding alert, continue monitoring from the start of process 1;
14. If the body angle does exceed the lying state threshold then the person was not able to rise unaided, raise a corresponding alarm.
   Process 2
1. The root mean square value of the signal is computed every minute;
2. The root mean square is compared to a threshold value;
3. If the root mean square exceeds the value of the threshold then continue monitoring from the start of process 2;
4. If the root mean square is less than the threshold value then continue monitoring the next samples for a threshold period of time (6 hours);
5. If the value of the root mean square exceeds the threshold value at any time during the 6 hour period then continue monitoring from the start of process 2;
6. If the value of the root mean square does not exceed the threshold value at any time during the 6 hour period then an extended absence of movement indicative of an inability to rise has been identified;
7. Raise a corresponding alarm.

In these embodiments alerts and alarms preferably are raised in the following manner:
1. Wireless communications are established between the wearable ambulatory monitor and a base-station that is able to connect to a telephone line;
2. The event is recorded and an electronic message is sent to a monitoring call centre, in the event of an alert, no further action is taken.
3. In the event of an alarm, the telephone line is opened by the base-station, one of three pre-programmed numbers is dialled and voice communications are established between the monitoring call centre and the person.

The invention has been described by way of example only with reference to preferred embodiments which is not intended to introduce limitations on the scope of the invention. It will be appreciated by persons skilled in the art that alternative embodiments exist even though they may not have been described herein which remain within the scope and spirit of the invention as broadly described herein.

1. A monitoring apparatus for an ambulatory subject including:
a portable monitor mountable on the subject that includes an accelerometer that determines the instant acceleration of the subject in one or more determined directions;
a processing unit that:
a) determines at least one instant ambulatory performance indicia of the subject from at least one determined instant acceleration of the subject in one or more instants of time;
b) determines at least one designated performance threshold from at least one previously determined instant ambulatory performance indicia;
c) determines if the subject's instant ambulatory performance indicia is below or above the at least one designated performance threshold;
d) initiates at least one event if the determined instant ambulatory performance indicia is above or below the determined at least one designated performance threshold; and
a communications unit that communicates an initiated event to a remote receiver.

2. A monitoring apparatus in accordance with claim 1 wherein the at least one designated performance threshold is determined by the processing unit from a plurality of previously determined instant ambulatory performance indicia.

3. A monitoring apparatus in accordance with claim 1 wherein the at least one event is initiated only if the determined instant ambulatory performance indicia is below or above the determined at least one designated performance threshold for a designated period of time.

4. A monitoring apparatus in accordance with claim 3 wherein the designated first period of time is determined from a plurality of previously determined instant ambulatory performance indicia.

5. A monitoring apparatus in accordance with claim 1 wherein the accelerometer simultaneously determines the acceleration of the subject in three orthogonal directions.

6. A monitoring apparatus in accordance with claim 5 wherein the portable monitor is configured to be mounted on an upright ambulatory such that one of the three orthogonal directions is in a vertical direction or within a designated angle of the vertical direction.

7. A monitoring apparatus in accordance with claim 1 wherein an initiated event is communicated by the apparatus to the remote receiver by wireless communication.

8. A monitoring apparatus in accordance with claim 3 wherein:
a first instant ambulatory performance indicia representative of movement activity in the subject is determined from the instant magnitude of the sum of the instant acceleration of the subject in one or more determined directions;
a first acceleration threshold magnitude that is representative of a lack of normal expected subject movement is designated as a first designated performance threshold;
a first event representative of an absence of a normal amount of movement in the subject indicative of a possible inability to rise due to a collapse or other adverse event is initiated if the determined first instant ambulatory performance indicia is below the first designated acceleration threshold magnitude for a first designated period of time.

9. A monitoring apparatus in accordance with claim 8 wherein:
a second instant ambulatory performance indicia representative of the instant cranio-caudal angle of the subject relative to an upright disposition of the subject, is determined from at least one of the determined instant acceleration of the subject in one or more determined directions;
an angle magnitude that is representative of a cranio-caudal angle of the subject relative to an upright subject where the disposition of the subject is deemed to be no longer upright is designated as a second designated performance threshold;
a second acceleration threshold magnitude representative of an abnormally high subject movement is designated as a third designated performance threshold; and
a second event representative of an abnormal acceleration of the subject followed by a laying down subject disposition indicative of a possible fall coupled with a subsequent absence of getting up from the laying down disposition indicative of a possible debilitating fall is initiated if:
a) the determined first instant ambulatory performance indicia is above the second designated acceleration threshold for a second designated period of time; and
b) within a third designated period of time of the end of the second designated period of time the determined second instant ambulatory performance indicia is greater than the designated angle threshold; and then
c) the determined first instant ambulatory performance indicia is below the first designated acceleration threshold magnitude for a first designated period of time.

10. A method of monitoring an ambulatory subject including:
mounting a portable monitor on the subject that is in wireless communication with a remote receiver, wherein the monitor includes an accelerometer to determine simultaneously the instant acceleration of the subject in at least three different directions at different instants in time;
determining a plurality of instant ambulatory performance indicia based on the determined instant acceleration of the subject;
determining at least one designated performance threshold corresponding to the each ambulatory performance indicia from at least one previously determined corresponding instant ambulatory performance indicia;
determining if the subject's instant ambulatory performance indicia is below or above the corresponding at least one designated performance threshold and initiating a corresponding event;
communicating a designated initiated event to the remote receiver.

11. A method of monitoring an ambulatory subject in accordance with claim 10 wherein the at least one designated performance threshold is determined from a plurality of previously determined instant performance indicia, and the designated performance threshold can responsively and cooperatively adapt to statistical changes in previously determined instant performance indicia over time.

12. A method of monitoring an ambulatory subject in accordance with claim 10 wherein the event is initiated only if the determined instant ambulatory performance indicia is below or above the determined at least one designated performance threshold for a designated period of time.

13. A monitoring apparatus for an ambulatory subject including:
a portable monitor mountable on the subject that includes an accelerometer that simultaneously determines the instant acceleration of the subject in at least three different directions;
a processing unit that:
a) determines the instant magnitude of the sum of the instant acceleration of the subject in the at least three different directions;
b) determines if the determined instant magnitude does not exceed a first designated acceleration threshold magnitude for a first designated period of time, where the first designated acceleration threshold magnitude is representative of a lack of normal expected subject movement;
c) initiates an event representative of an absence of a normal amount of movement in the subject indicative of a possible inability to rise due to a collapse or other adverse event if the determined instant magnitude does not exceed the first designated acceleration threshold magnitude for at least the first designated period of time; and
a communications unit that communicates an initiated event to a remote receiver.

14. A monitoring apparatus in accordance with claim 13 wherein:
the processing unit:
a) determines if the determined instant magnitude of the sum of the instant acceleration of the subject in the at least three different directions exceeds a second designated acceleration threshold magnitude for at least a second designated period of time;
b) determines the magnitude of the instant angle of the subject being the magnitude of the angle between the cranio-caudal axis of the subject and the cranio-caudal axis of the subject when in an upright disposition from at least one of the determined instant acceleration of the subject in one or more determined directions;
c) determines if the determined instant angle of the monitor is greater or less than a designated angle magnitude threshold; and
d) initiates an event representative of an abnormally high acceleration of the subject followed by a laying down subject disposition indicative of a possible fall coupled with a subsequent absence of getting up from the laying down disposition indicative of a possible debilitating fall if:
I. the determined instant magnitude of the sum of the instant acceleration of the subject in the at least three different directions exceeds the second designated acceleration threshold magnitude for at least a second designated period of time; and
II. within a third designated period of time after the end of the second designated period of time the determined instant angle of the subject is greater than the designated angle magnitude threshold; and
III. within the third designated period of time after the end second designated period of time the instant magnitude of the sum of the instant acceleration of the subject in the at least three different directions does not exceed a third designated acceleration magnitude for at least a fourth designated period of time.

15. A monitoring apparatus in accordance with claim 13 wherein the accelerometer simultaneously determines the acceleration of the subject in three orthogonal directions.

16. A monitoring apparatus in accordance with claims 14 wherein the portable monitor is mounted on an upright subject in an orientation so that one of the three orthogonal directions is in a vertical direction or within a designated angle of the vertical direction.
